(19) United States
(12) Patent Application Publication
(10) Pub. No.: US 2005/0171567 A1
(43) Pub. Date: Aug. 4, 2005

(54) Lancet and method of manufacturing the same

(76) Inventor: Damon H. DeHart, Bedford, MA (US)

Correspondence Address: KRIEGSMAN & KRIEGSMAN, 665 Franklin Street, Framingham, MA, 01702, US

(21) Appl. No.: 10/767,867

(22) Filed: Jan. 29, 2004

(51) Int. Cl. A61B 17/14; A61B 17/32

(52) U.S. Cl. 606/181

(57) ABSTRACT

A lancet for acquiring a blood sample includes a unitary member which is hollow along at least a portion of its length. Preferably, the unitary member is in the form of a seamless cylindrical tube which is constructed out of a rigid and durable material, such as stainless steel. During the lancet manufacturing process, a series of individual grinding processes are performed on one end of the unitary member to yield a lancet which includes a plurality of skin-penetrable tips. In one embodiment, one end of the unitary member is subjected to a pair of single angle planar grinds to yield a lancet which includes a pair of sharpened tips. In another embodiment, one end of the unitary member is subjected to four compound angle planar grinds to yield a lancet which includes a pair of sharpened tips. In another embodiment, one end of the unitary member is subjected to three single angle planar grinds to yield a lancet which includes three equidistantly spaced sharpened tips.

### BACKGROUND OF THE INVENTION

The present invention relates generally to lancets and more particularly to a novel lancet and method of manufacturing the same.

Diabetes is a disease which typically requires a patient to routinely measure the concentration of glucose in his/her blood. Based upon the results of each blood glucose measurement, the patient may require a particular drug treatment (e.g., an injection of insulin) in order to regulate that the blood glucose level of the patient remains within a specified range. Exceeding the upper limit of said range (hyperglycemia) or dropping beneath the lower limit of said range (hypoglycemia) should be avoided with as much diligence as possible to prevent the patient from experiencing serious medical complications which include, inter alia, retinopathy, nephropathy, and neuropathy.

A two-step process is commonly practiced by diabetes patients to self-monitor the level of glucose present in their blood. In the first step, the patient makes a skin prick (typically in his/her finger) in order to acquire a small sample of blood. In the second step, a blood glucose monitor is used to calculate and, in turn, digitally display the concentration of glucose present in the blood sample.

Blood samples taken from a patient for blood glucose monitoring are typically obtained by piercing the skin of the patient using a lancet. A lancet is designed to penetrate through the epidermis (the outermost layer of the skin) of the patient and into the dermis (the layer of skin directly beneath the epidermis) which is replete with capillary beds. The puncture of one or more capillaries by the lancet generates a sample of blood which exits through the incision in the skin.

Lancets are commonly constructed by cutting a solid, elongated length of cylindrical wire at specified intervals to generate a plurality of shortened wire samples of identical size and shape. The plurality of individual wire samples are mounted parallel to one another within a single cassette. The cassette is then inserted into a grinding machine with the first end of each wire sample directed towards a grinding wheel. In what is typically referred to as a grinding process (or simply a grind), the cassette is positioned in a particular orientation and drawn into contact against the grinding wheel. Subsequent thereto, the cassette is commonly repositioned in different orientations and drawn back into contact against the grinding wheel in additional grinding processes. All in all, three separate grinds are commonly performed on the plurality of individual wire samples to create at the first end of each wire sample a sharpened tip with two separate cutting edges. Upon completion of the grinding processes, the second end of each lancet is often embedded within an enlarged plastic base (e.g., through the process of insert molding) to facilitate its handling.

Lancets of the type as described above are often fired into the skin of a patient to draw a blood sample using a lancing device. A lancing device typically includes a holder into which the lancet may be permanently or removably mounted. A spring-loaded firing mechanism is traditionally coupled to the lancet holder. The firing mechanism commonly includes some type of actuation means, such as an externally-accessible button, which when actuated fires the lancet holder towards the patient such that sharpened tip of the lancet penetrates the skin of the patient.

Conventional lancets typically include a single sharpened tip (with two cutting edges) which is adapted to penetrate into the skin of the patient. However, it has been found that lancets which include a single sharpened tip often suffer from a couple notable drawbacks.

As a first drawback, lancets which include a single sharpened tip often fail to puncture a significant number of capillaries located within the dermis layer of the patient's skin. As a result, multiple firings of the lancet may be required in order to produce an adequate blood sample, the additional lancet firings increasing the overall discomfort experienced by the patient, which is highly undesirable.

As a second drawback, lancets which include a single sharpened tip are often constructed with a sharpened tip of increased diameter to insure that the tip punctures at least one capillary upon insertion into the dermis layer of the skin. However, the utilization of a lancet which includes a sharpened tip of increased diameter causes said lancet to contact a greater number of nerve endings in the skin, thereby increasing patient discomfort, which is highly undesirable.

Accordingly, lancets constructed to include a plurality of reduced diameter tips are known in the art. Lancets which include a plurality of reduced diameter tips have a greater probability of penetrating a capillary upon insertion, thereby increasing the likelihood of producing an adequate blood sample. Furthermore, lancets which include a plurality of reduced diameter tips create relatively small incision wounds in the skin, thereby minimizing patient discomfort, which is highly desirable.

In U.S. Pat. No. 2,801,633 which issued on Aug. 6, 1957 in the name of J. C. Ehrlich, there is disclosed, in one embodiment, a lancet comprising two skin-penetrating elements. The lancet is constructed from a blank of flexible sheet metal of a thickness no greater than about 0.0025 inches. The blank is provided with two point members which project from a common edge. The blank is rolled into a tube which has considerable rigidity despite the thinness of the metal stock. With the blank rolled into a tube, the two points project out from the same end of the tube (the two points may be diametrically opposite each other). The pair of points serves to create two puncture sites in the skin of the patient which, in turn, insures a substantial flow of blood from the patient without the necessity for squeezing the area of puncture.

The lancet described in U.S. Pat. No. 2,801,633 to Ehrlich is constructed by first stamping a flat sheet of metal to include a pair of sharpened, generally V-shaped tips, both of said tips protruding out from a first edge of the flat sheet of metal. Upon completion of the stamping step, the flat sheet of metal is rolled into a tubular shape to create a hollow cylindrical needle with the pair of sharpened tips extending out from one end. The flat sheet of material is maintained in its tubular shape by welding together second and third edges of the flat sheet of metal, said welding process creating a longitudinal seam which extends along the majority of the length of the lancet.

The multi-tip lancet described in U.S. Pat. No. 2,801,633 to Ehrlich suffers from a notable shortcoming. Specifically, as noted above, this type of multi-tip lancet is traditionally manufactured using separate stamping, rolling and welding processes. As can be appreciated, the manufacture of a lancet using separate stamping, rolling and welding processes increases the complexity and overall cost of the manufacturing process, which is highly undesirable.

### SUMMARY OF THE INVENTION

It is an object of the present invention to provide a novel lancet.

It is another object of the present invention to provide a lancet of the type described above which can be used to extract an adequate amount of blood for testing purposes with minimal discomfort to the patient.

It is yet another object of the present invention to provide a simple and inexpensive method of manufacturing the lancet as described above.

Therefore, according to one feature of the present invention, there is provided a lancet comprising a seamless unitary member which is hollowed along at least a portion of its length, said unitary member including a first end, a second end, and a longitudinal axis, wherein the first end of said unitary member is shaped to include first and second sharpened tips.

According to another feature of the present invention, there is provided a lancet comprising a unitary member which is hollowed along at least a portion of its length, said unitary member including a first end and a second end, wherein the first end of said unitary member includes first and second ground surfaces which at least partially define first and second sharpened tips.

According to another feature of the present invention, there is provided a method of manufacturing a lancet, said method comprising the steps of providing a unitary member, said unitary member including a first end and a second end, and performing first and second grinds on the first end of said unitary member so as to yield first and second ground surfaces in the first end of said unitary member, said first and second ground surfaces at least partially defining first and second sharpened tips.

According to another feature of the present invention, there is provided a lancet comprising a unitary member which is hollowed along at least a portion of its length, said unitary member including a first end and a second end, wherein the first end of said unitary member is shaped to include first, second and third sharpened tips.

According to another feature of the present invention, there is provided a method of manufacturing a lancet, said method comprising the steps of providing a unitary member, said unitary member including a first end and a second end, and performing first, second and third grinds on the first end of said unitary member so as to yield first, second and third ground surfaces in the first end of said unitary member, said first, second and third ground surfaces at least partially defining first, second and third sharpened tips in said unitary member.

According to another feature of the present invention, there is provided a lancet comprising a unitary member including a first end, a second end and a longitudinal axis, wherein the first end of said unitary member is shaped to include first, second and third ground surfaces which together at least partially define a single tip and first, second and third cutting edges.

According to another feature of the present invention, there is provided a method of manufacturing a lancet, said method comprising the steps of providing a unitary member which includes a first end, a second end and a longitudinal axis, and performing three separate grinds on the first end of said unitary member to yield first, second and third cutting edges which at least partially define a single sharpened tip.

According to another feature of the present invention, there is provided a lancet comprising a unitary member including a first end, a second end and a longitudinal axis, wherein the first end of said unitary member is shaped to include first, second, third and fourth ground surfaces which together at least partially define a single tip and first, second, third and fourth cutting edges.

According to another feature of the present invention, there is provided a lancet comprising a method of manufacturing a lancet, said method comprising the steps of providing a unitary member which includes a first end, a second end and a longitudinal axis, and performing four separate grinds on the first end of said unitary member to yield first, second, third and fourth cutting edges which at least partially define a single sharpened tip.

Various other features and advantages will appear from the description to follow. In the description, reference is made to the accompanying drawings which form a part thereof, and in which is shown by way of illustration, various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is therefore, not to be taken in a limiting sense, and the scope of the present invention is best defined by the appended claims.

### BRIEF DESCRIPTION OF THE DRAWINGS

In the drawings wherein like reference numerals represent like parts:

FIGS. 1(a)-(d) represent perspective, top, front and right end views, respectively, of a first embodiment of a lancet constructed according to the teachings of the present invention;

FIG. 2 is perspective view of a unitary member which is subject to series of grinding processes to form the lancet shown in FIGS. 1(a)-(d);

FIGS. 3(a)-(d) represent perspective, top, front and right side views, respectively, of a second embodiment of a lancet constructed according to the teachings of the present invention;

FIGS. 4(a)-(d) represent perspective, top, front and right side views, respectively, of a third embodiment of a lancet constructed according to the teachings of the present invention;

FIGS. 5(a)-(d) represent perspective, top, front and right side views, respectively, of a fourth embodiment of a lancet constructed according to the teachings of the present invention;

FIGS. 6(a)-(d) represent perspective, top, front and right side views, respectively, of a fifth embodiment of a lancet constructed according to the teachings of the present invention;

FIGS. 7(a)-(d) represent perspective, top, front and right side views, respectively, of a sixth embodiment of a lancet constructed according to the teachings of the present invention;

FIGS. 8(a)-(d) represent perspective, top, front and right side views, respectively, of a seventh embodiment of a lancet constructed according to the teachings of the present invention; and

FIGS. 9(a)-(e) represent perspective, front, top, right side and enlarged fragmentary front views, respectively, of an eighth embodiment of a lancet constructed according to the teachings of the present invention.

### DETAILED DESCRIPTION OF THE PREFERRED EMBODIMENT

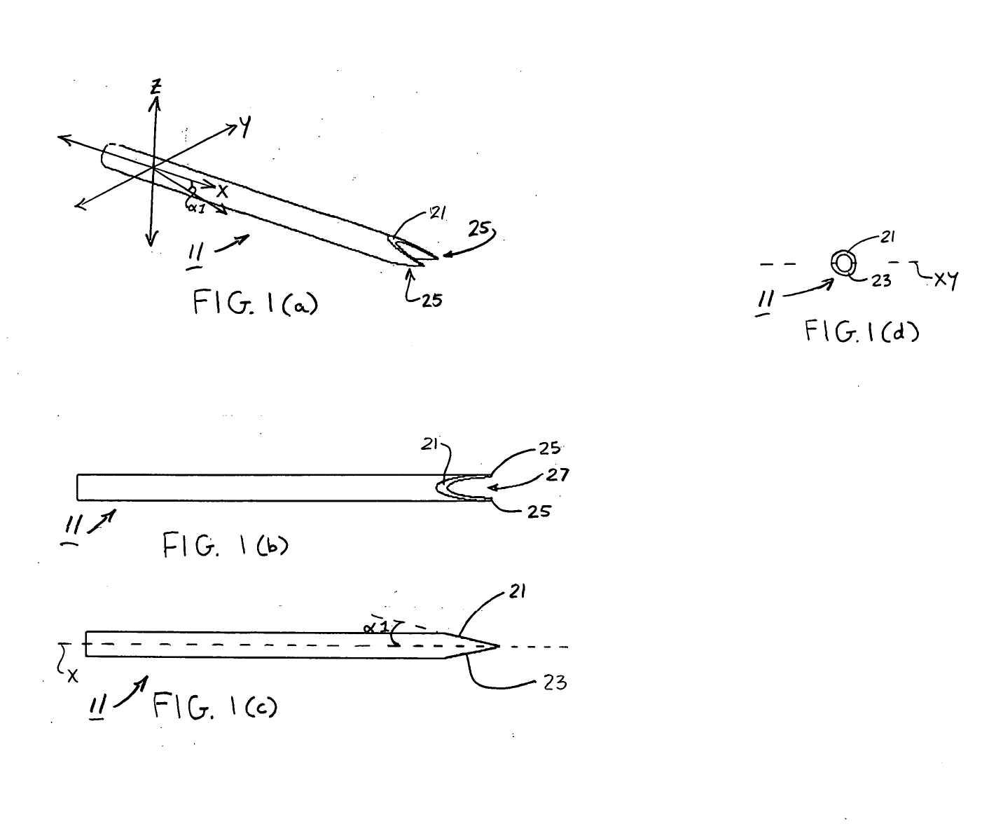

Referring now to the drawings, there is shown in FIGS. 1(a)-(d), a first embodiment of a lancet which is constructed according to the teachings of the present invention, the lancet being identified generally by reference numeral 11. As can be appreciated, lancet 11 is designed to be fired into the skin of a patient in order to acquire a blood sample for testing purposes.

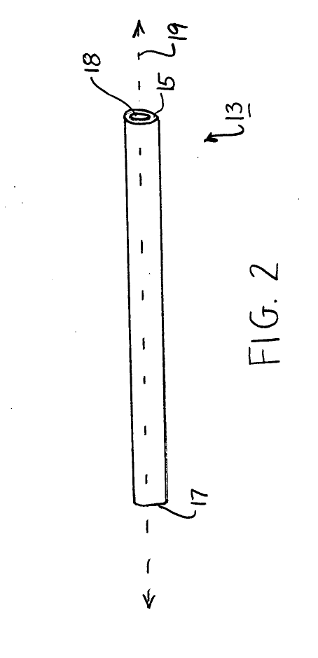

Referring now to FIG. 2, lancet 11 is preferably formed by subjecting a unitary member 13 to a series of grinding processes which will be described further in detail below. Unitary member 13 is preferably constructed of a 304 gage, full hard, stainless steel material to ensure that lancet 11 will have the necessary rigidity to function properly. Unitary member 13 is also preferably constructed of a stainless steel material which is treated with a bright finish to maximize the sharpness of the skin-penetrable tips which are created through the series of grinding processes.

It should be noted that unitary member 13 is not limited to being manufactured of a rigid stainless steel material. Rather, it is to be understood that unitary member 13 could be manufactured out of any suitable material (e.g., a ceramic material) which has an adequate amount of rigidity and which could be ground to form a plurality of sharpened tips without departing from the spirit of the present invention.

Unitary member 13 is represented herein as being in the form of an elongated, seamless, cylindrical tube which is hollow along its length. As a result, unitary member 13 has a lateral cross-section which is annular and uniform along its length. Unitary member 13 includes a first end 15, a second end 17, a longitudinal bore 18 and a longitudinal axis 19. Due to its uniform lateral cross-section, unitary member 13 can be mass produced by cutting an elongated, hollow cylindrical tube at equidistantly spaced intervals, which is highly desirable.

It should be noted that unitary member 13 is not limited to being in the form of an elongated cylindrical tube which is hollow along its length. Rather, it is to be understood that unitary member 13 could have a different shape (e.g., an oval-shaped lateral cross-section) without departing from the spirit of the present invention. Furthermore, it is to be understood that unitary member 13 need not be hollow along its length. Rather, unitary member 13 could be partially hollowed along its length (i.e., at first end 15 only) or solid along its entire length (and at least partially hollowed out in a subsequent boring, or drilling, process) without departing from the spirit of the present invention.

Referring back to FIG. 1(a), lancet 11 is shown in relation to its longitudinal axis X, its lateral axis Y and its vertical axis Z. Together, longitudinal axis X and lateral axis Y define a horizontal plane XY.

As noted briefly above, lancet 11 is formed by subjecting first end 15 of unitary member 13 to a series of grinding processes. Specifically, as seen most clearly in FIGS. 1(a)-(d), a first grinding process is performed on first end 15 of unitary member 13 so as to create a first ground surface 21. As seen most clearly in FIGS. 1(a) and 1(c), first ground surface 21 is a planar surface which extends from horizontal plane XY at an angle al of approximately 5 degrees relative to longitudinal axis X. First ground surface 21 is referred to in the art as a single angle ground surface because ground surface 21 extends from horizontal plane at a single angle relative thereto (i.e., at an angle relative to a single axis).

As seen most clearly in FIG. 1(d), the first grinding process generates a first ground surface 21 in first end 15 of unitary member 13 which extends approximately 180 degrees about longitudinal axis X. Accordingly, after said first grind, unitary member 13 (with first ground surface 21 formed therein) is rotated 180 degrees about longitudinal axis X and its first end 15 (with first ground surface 21 formed therein) is subjected to a secondary grinding process which is identical in nature to the primary grinding process. As a result of said second grinding process, a second ground surface 23 is formed which mirrors first grinding surface 23 about horizontal plane XY. Completion of the secondary grind results in finished lancet 11.

As can be seen, together the first and second grinding processes create a pair of identically-shaped, sharpened tips, or points, 25 which are spaced 180 degrees apart from one another. As seen most clearly in FIG. 1(b), tips 25 define a gullet 27 therebetween which is generally U-shaped in lateral cross-section.

It should be noted that the angle α1 at which ground surfaces 21 and 23 are formed could be modified without departing from the spirit of the present invention. As can be appreciated, increasing the value of angle α1 would render the resulting lancet tips more robust (i.e., firm and strong) but, at the same time, less sharp. Similarly, decreasing the value of angle α1 would render the resulting lancet tips more sharp but, at the same time, more flimsy (i.e., infirm). As such, the value of angle α1 could be adjusted by the lancet manufacturer to meet specific lancet performance needs.

Upon completion of its manufacturing, the end of lancet 11 opposite tips 25 is preferably embedded into a plastic base (not shown) such as through a process of insert molding. As can be appreciated, a plastic base would serve to improve handling and/or mounting requirements for lancet 11.

In use, lancet 11 can be used to draw a blood sample from a patient in the following manner. Specifically, lancet 11 is orientated such that sharpened tips 25 are directed towards the sample site on the patient. Lancet 11 is then driven (e.g., using a mechanical lancet device) such that both sharpened tips 25 penetrate into the dermis layer of the patient's skin and puncture at least one capillary therein. Preferably, lancet 11 is driven a distance which is less than the length of gullet 27 to minimize the surface area of lancet 11 which penetrates into the patient's skin, thereby minimizing patient discomfort. Lancet 11 is then withdrawn from the patient which causes a blood sample to exit the wound site, said blood sample being available for testing purposes.

The particular design of lancet 11 introduces a notable advantage over conventional lancets which include only a single tip. Specifically, the fact that lancet 11 includes a pair of sharpened tips 25 (as opposed to a single tip as found in most conventional lancets) serves to increase the probability of lancet 11 penetrating a capillary when inserted in the dermis layer of a patient, which is a principal object of the present invention. Furthermore, because two separate tips 25 are formed, the cross-sectional diameter of each tip 25 can be decreased to minimize patient discomfort during lancing, which is another principal object of the present invention.

It should be noted that numerous design modifications could be made to lancet 11 without departing from the spirit of the present invention. In particular, modifications to the quantity and relative sharpness of tips 25 could be made to lancet 11 without departing from the spirit of the present invention, as will be described further below.

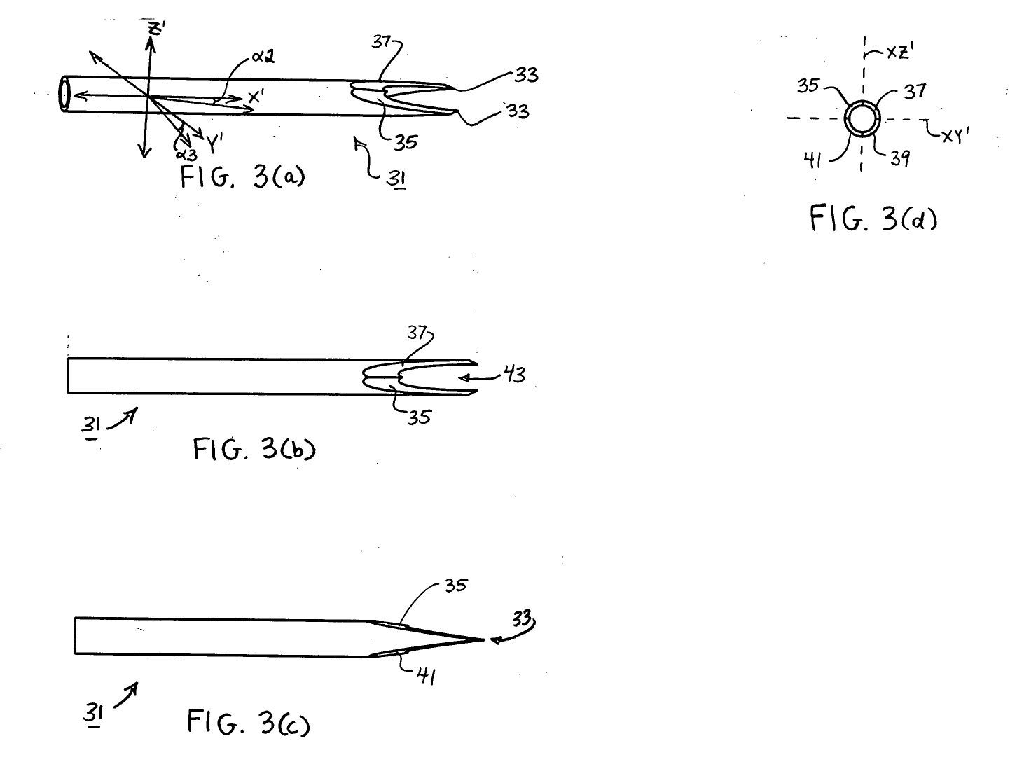

Referring now to FIGS. 3(a)-(d), there is shown a second embodiment of a lancet constructed according to the teachings of the present invention, the lancet being identified generally by reference numeral 31. Lancet 31 is similar to lancet 11 in that lancet 31 includes a pair of sharpened tips 33. However, lancet 31 differs from lancet 11 in that lancet tips 33 are generated through a series of four (rather than two) grinding processes. As a result of the two additional grinding processes, lancet 31 is provided with tips 33 which are sharper in construction than tips 25 of lancet 11.

In FIG. 3(a), lancet 31 is shown in relation to its longitudinal axis X′, its lateral axis Y′ and its vertical axis Z′. Together longitudinal axis X′ and lateral axis Y′ define a horizontal plane XY′. In addition, together longitudinal axis X′ and vertical axis Z′ define a vertical plane XZ′.

Lancet 31 is similar to lancet 11 in that lancet 31 is formed from unitary member 13. In order to manufacture lancet 31, first end 15 of unitary member 13 is subjected to four separate grinding processes. Specifically, a first grinding process is performed on first end 15 of unitary member 13 so as to create a first ground surface 35. As seen most clearly in FIG. 3(a), first ground surface 35 is a substantially planar surface which extends from horizontal plane XY′ at both an angle α2 of approximately 7 degrees relative to longitudinal axis X′ and an angle α3 of approximately 15 degrees relative to lateral axis Y′. It should be noted that first ground surface 35 is referred to in the art as a compound angle ground surface (or simply as a compound ground surface) because ground surface 35 is a planar surface which extends from horizontal plane XY′ at multiple angles relative thereto (i.e., at an angle relative to more than one axis).

As seen most clearly in FIG. 3(d), the first grinding process generates a first ground surface 35 in first end 15 of unitary member 13 which extends approximately 90 degrees about longitudinal axis X′. Accordingly, after said first grind, unitary member 13 (with first ground surface 35 formed therein) is rotated 90 degrees in the counterclockwise direction about longitudinal axis X and is subjected to a second grinding process. As a result of said second grinding process, a second compound angle ground surface 37 is formed which mirrors first grinding surface 35 about vertical plane XZ′. After said second grind, unitary member 13 (with first and second ground surfaces 35 and 37 formed therein) is rotated another 90 degrees in the counterclockwise direction about longitudinal axis X and is subject to a third grinding process. As a result of said third grinding process, a third compound angle ground surface 39 is formed which mirrors second ground surface 37 about horizontal plane XY′. After said third grind, unitary member 13 (with first, second and third ground surfaces 35, 37 and 39 formed therein) is rotated another 90 degrees in the counterclockwise direction about longitudinal axis X and is subjected to a fourth grinding process. As a result of said fourth grinding process, a fourth compound angle ground surface 41 is formed which mirrors first ground surface 35 about horizontal plane XY′. Completion of the fourth grinding process produces finished lancet 31.

As can be seen, together the first, second, third and fourth grinding processes create a pair of skin-penetrable sharpened tips, or points, 33 which are spaced 180 degrees apart from one another. As seen most clearly in FIG. 3(b), tips 33 define a gullet 43 therebetween which is generally U-shaped in lateral cross-section.

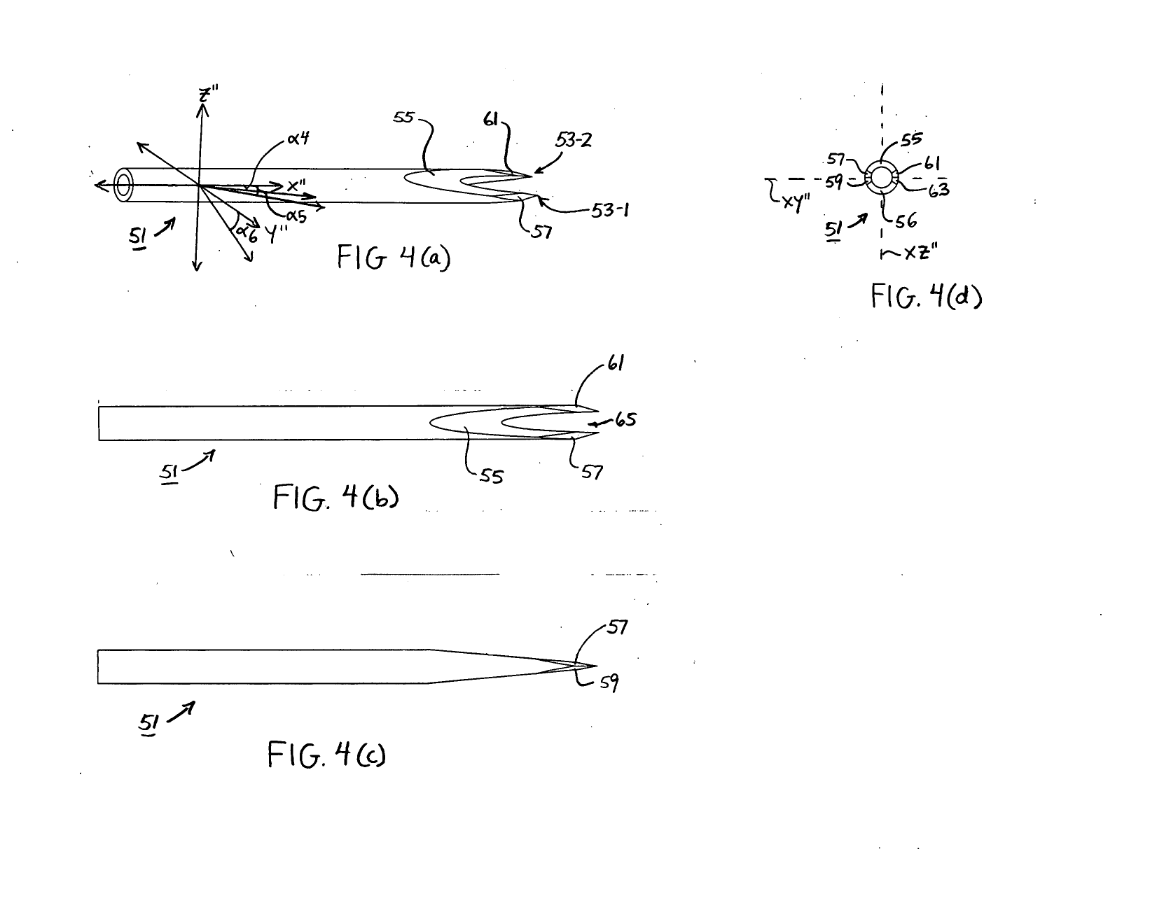

Referring now to FIGS. 4(a)-(d), there is shown a third embodiment of a lancet constructed according to the teachings of the present invention, the lancet being identified by reference numeral 51. Lancet 51 is similar to lancet 11 in that lancet 51 includes a pair of sharpened tips 53. However, lancet 51 differs from lancet 11 in that lancet tips 53 are generated through a series of six (rather than two) grinding processes. As a result of the four additional grinding processes, lancet 51 is provided with tips 53 which are sharper in construction than tips 25 of lancet 11.

In FIG. 4(a), lancet 51 is shown in relation to its longitudinal axis X″, its lateral axis Y″ and its vertical axis Z″. Together longitudinal axis X″ and lateral axis Y″ define a horizontal plane XY″. In addition, together longitudinal axis X″ and vertical axis Z″ define a vertical plane XZ″.

Lancet 51 is similar to lancet 11 in that lancet 51 is formed from unitary member 13. In fact, lancet 51 is formed by performing four additional grinding operations to lancet 11. Specifically, in order to manufacture lancet 51, a first grinding process is performed on first end 15 of unitary member 13 so as to create a first ground surface 55. As seen most clearly in FIG. 4(a), first ground surface 55 is a planar surface which extends from horizontal plane XY″ at an angle α4 of approximately 5 degrees relative to longitudinal axis X″. As can be appreciated, first grinding process produces a first ground surface 55 in first end 15 of unitary member 13 which extends approximately 180 degrees about longitudinal axis X″. Accordingly, after said first grind, unitary member 13 (with first ground surface 55 formed therein) is rotated 180 degrees about longitudinal axis X″ and its first end 15 (with first ground surface 55 formed therein) is subjected to a secondary grinding process which is identical in nature to the primary grinding process. As a result of said second grinding process, a second ground surface 56 is formed which mirrors first grinding surface 55 about horizontal plane XY″. Completion of the second grinding process produces lancet 11.

Lancet 11 is then subjected to four additional grinding processes. Specifically, a third grinding process is performed on one tip 25 of lancet 11 to create a third ground surface 57. Third ground surface 57 is a compound angle ground surface which is planar and which extends from horizontal plane XY″ at both an angle α5 of approximately 8 degrees relative to longitudinal axis X″ and an angle α6 of approximately 30 degrees relative to lateral axis Y″. The completion of the third grinding process creates a third ground surface 57 which extends approximately 180 degrees about one of said tips 25. Subsequent thereto, a fourth grinding process is performed on the same tip 25 of lancet 11. As a result of said fourth grinding process, a fourth compound angle ground surface 59 is formed on the same tip, said fourth ground surface 59 mirroring third ground surface 57 about horizontal plane XY′, as seen most clearly in FIGS. 4(c) and (d).

A fifth grinding process is performed on the other tip 25 of lancet 11 to create a fifth ground surface 61. As seen most clearly in FIGS. 4(b) and (d), fifth ground surface 61 is a compound angle ground surface which mirrors third ground surface about vertical plane XZ″. After the fifth grinding process, a sixth grinding process is performed. As a result of the sixth grinding process, a sixth compound angle ground surface 63 is formed which mirrors fifth compound angle ground surface 61 about horizontal plane XY″.

As can be seen, together the six grinding processes create a pair of sharpened tips 53-1 and 53-2 which are spaced 180 degrees apart from one another about longitudinal axis X″. As seen most clearly in FIG. 4(b), tips 53 define a gullet 65 therebetween which is generally U-shaped in lateral cross-section.

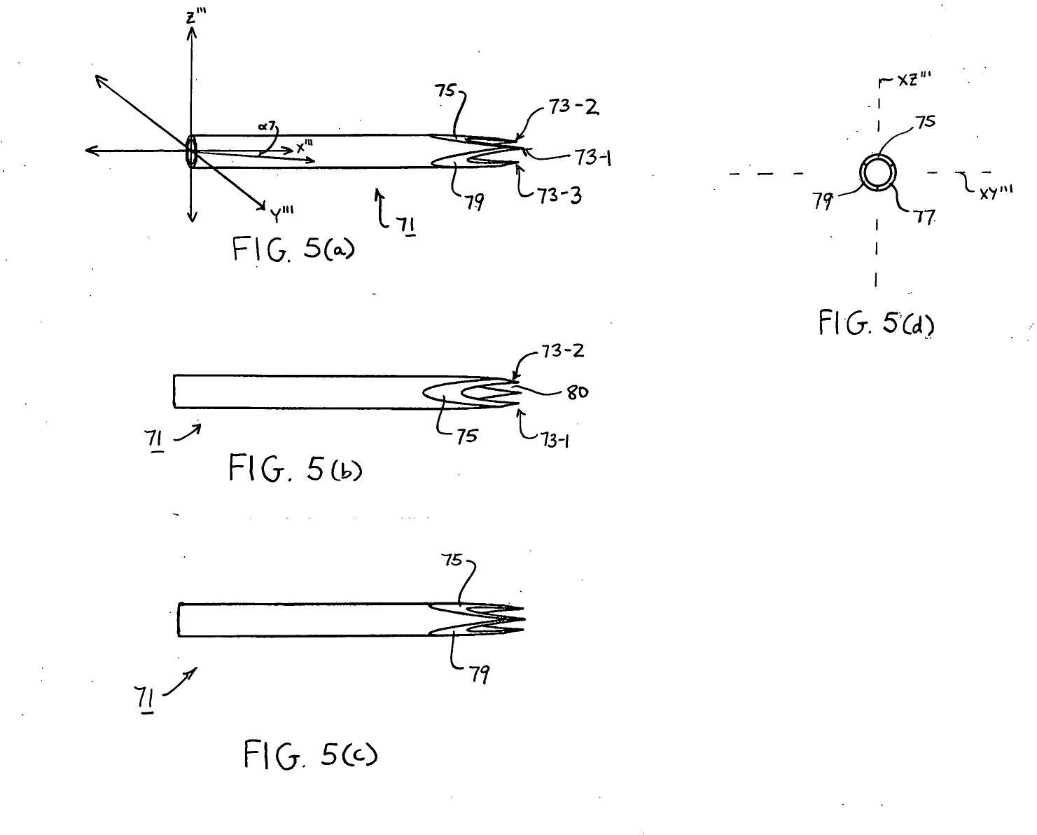

Referring now to FIGS. 5(a)-(d), there is shown a fourth embodiment of a lancet constructed according to the teachings of the present invention, the lancet being identified generally by reference numeral 71. Lancet 71 differs from lancet 11 primarily in that lancet 71 comprises three sharpened tips 73 (whereas lancet 11 includes only a pair of sharpened tips 25) which are created from three separate grinding processes (whereas lancet 11 is created from only a pair of grinding processes). It should be noted that the fact that lancet 71 includes three (rather than two) sharpened tips 73 improves the probability that lancet 71 will puncture a capillary when fired, which is a principal object of the present invention.

In FIG. 5(a), lancet 71 is shown in relation to its longitudinal axis X′″, its lateral axis Y′″ and its vertical axis Z′″. Together longitudinal axis X′″ and lateral axis Y′″ define a horizontal plane XY′″. In addition, together longitudinal axis X′″ and vertical axis Z′″ define a vertical plane XZ′″.

Lancet 71 is similar to lancet 11 in that lancet 71 is formed from unitary member 13. To manufacture lancet 71, first end 15 of unitary member 13 is subjected to three separate grinding processes. Specifically, a first grinding process is performed on first end 15 of unitary member 13 so as to create a first single angle ground surface 75. As seen most clearly in FIGS. 5(a) and 5(b), first ground surface 75 is a planar surface which extends from horizontal plane XY′″ at an angle α7 of approximately 7 degrees relative to longitudinal axis X′″.

As seen most clearly in FIG. 5(d), the first grinding process generates a first ground surface 75 in first end 15 of unitary member 13 which extends approximately 120 degrees about longitudinal axis X′″. Accordingly, after said first grind, first end 15 of unitary member 13 (with first ground surface 75 formed therein) is rotated 120 degrees in the counterclockwise direction about longitudinal axis X′″ and is subjected to a second grinding process which is identical in nature to the first grinding process. As a result of the second grinding process, a second single angle ground surface 77 is formed. Furthermore, after said second grind, first end 15 of unitary member 13 (with first and second ground surfaces 75 and 77 formed therein) is rotated an additional 120 degrees in the counterclockwise direction about longitudinal axis X′″ and is subjected to a third grinding process which is identical in nature to the first and second grinding processes. As a result of the third grinding process, a third single angle ground surface 79 is formed which mirrors second ground surface 77 about vertical plane XZ′″. Together, the three grinding processes create three sharpened tips 73-1, 73-2 and 73-3 which are spaced 120 degrees apart from one another about longitudinal axis X′″, adjacent tips 73 defining a gullet 80 therebetween which is generally U-shaped in lateral cross-section.

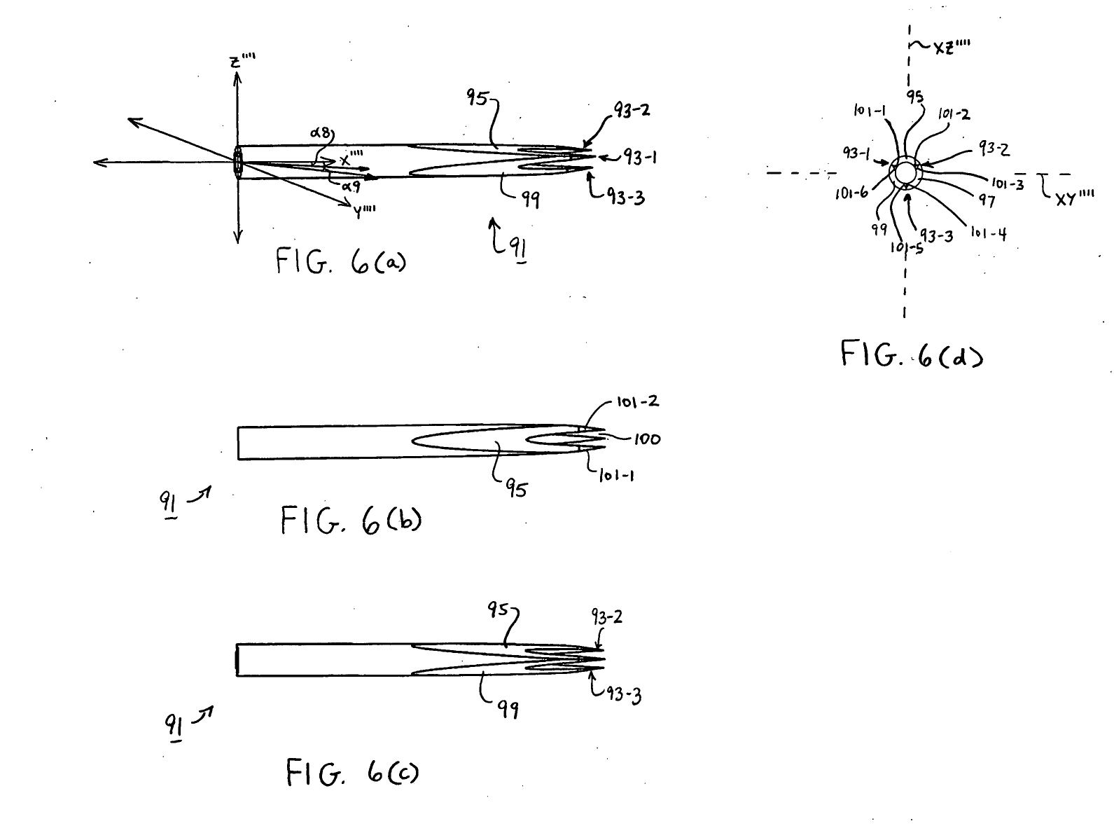

Referring now to FIGS. 6(a)-(d), there is shown a fifth embodiment of a lancet constructed according to the teachings of the present invention, the lancet being identified generally by reference numeral 91. Lancet 91 is similar to lancet 71 in that lancet 91 includes three sharpened tips 93. However, lancet 91 differs from lancet 71 in that lancet 91 is created by subjecting unitary member 13 to six separate grinding processes (whereas lancet 71 is created by subjecting unitary member 13 to three separate grinding processes). It should be noted that the fact that lancet 91 is created by subjecting unitary member 13 to three more grinding processes than lancet 71 results in tips 93 of lancet 91 being stronger (i.e., robust) than tips 73 of lancet 71, as will be described further below.

In FIG. 6(a), lancet 91 is shown in relation to its longitudinal axis X″″, its lateral axis Y″″ and its vertical axis Z″″. Together longitudinal axis X″″ and lateral axis Y″″ define a horizontal plane XY″″. In addition, together longitudinal axis X″″ and vertical axis Z″″ define a vertical plane XZ″″.

The first three grinding processes in manufacturing lancet 91 are identical to the first three grinding processes in manufacturing lancet 71 with the exception of the particular grinding angle performed. Specifically, a first grinding process is performed on first end 15 of unitary member 13 so as to create a first single angle ground surface 95. As seen most clearly in FIGS. 6(a) and 6(b), first ground surface 95 is a planar surface which extends from horizontal plane XY″″ at an angle α8 of approximately 3 degrees relative to longitudinal axis X″″.

As seen most clearly in FIG. 6(d), the first grinding process generates a first ground surface 95 in first end 15 of unitary member 13 which extends approximately 120 degrees about longitudinal axis X″″. Accordingly, after said first grind, first end 15 of unitary member 13 (with first ground surface 95 formed therein) is rotated 120 degrees in the counterclockwise direction about longitudinal axis X″″ and is subjected to a second grinding process which is identical in nature to the first grinding process. As a result of the second grinding process, a second single angle ground surface 97 is formed. Furthermore, after said second grind, first end 15 of unitary member 13 (with first and second ground surfaces 95 and 97 formed therein) is rotated an additional 120 degrees in the counterclockwise direction about longitudinal axis X″″ and is subjected to a third grinding process which is identical in nature to the first and second grinding processes. As a result of the third grinding process, a third single angle ground surface 99 is formed which mirrors second ground surface 97 about vertical plane XZ″″. Together, the three grinding processes create three sharpened tips 93-1, 93-2 and 93-3 which are spaced 120 degrees apart from one another about longitudinal axis X″″, adjacent tips 93 defining a gullet 100 therebetween which is generally U-shaped in lateral cross-section.

Upon completion of the first three grinding processes, three additional grinding processes are performed which serve to shorten the length of each tip 93 and thereby increase its strength, which is highly desirable. Specifically, a fourth grinding process is performed with the unitary member disposed in the same orientation in which the first grinding process is performed. The fourth grinding process creates a pair of single angle ground surfaces 101-1 and 101-2 on tips 93-1 and 93-2, repsectively. As seen most clearly in FIG. 6(a) and 6(b), single angle ground surfaces 101-1 and 101-2 are planar surfaces which extend from horizontal plane XY″″ at an angle α9 of approximately 5 degrees relative to longitudinal axis X″″.

After said fourth grind, a fifth grinding process is performed with the unitary member disposed in the same orientation in which the second grinding process is performed. The fifth grinding process creates a pair of single angle ground surfaces 101-3 and 1014 on tips 93-2 and 93-3, respectively. Similarly, after said fifth grind, a sixth grinding process is performed with the unitary member disposed in the same orientation in which the third grinding process is performed. The sixth grinding process creates a pair of single angle ground surfaces 101-5 and 101-6 on tips 93-3 and 93-1, respectively. As can be appreciated, the fourth, fifth and sixth grinding processes serve to decrease the length of tips 93 (thereby rendering them more robust) while maintaining their sharpness, which is highly desirable.

The various grinding processes described above in conjunction with the multi-tip lancets of the present invention could be used to create a single tip lancet with an increased number of cutting edges as compared to conventional prior art lancets (which typically include two cutting edges). A single tip lancet which includes a greater number of cutting edges than a conventional lancet would be more likely to draw a blood sample when fired into the skin of a patient, which is highly desirable.

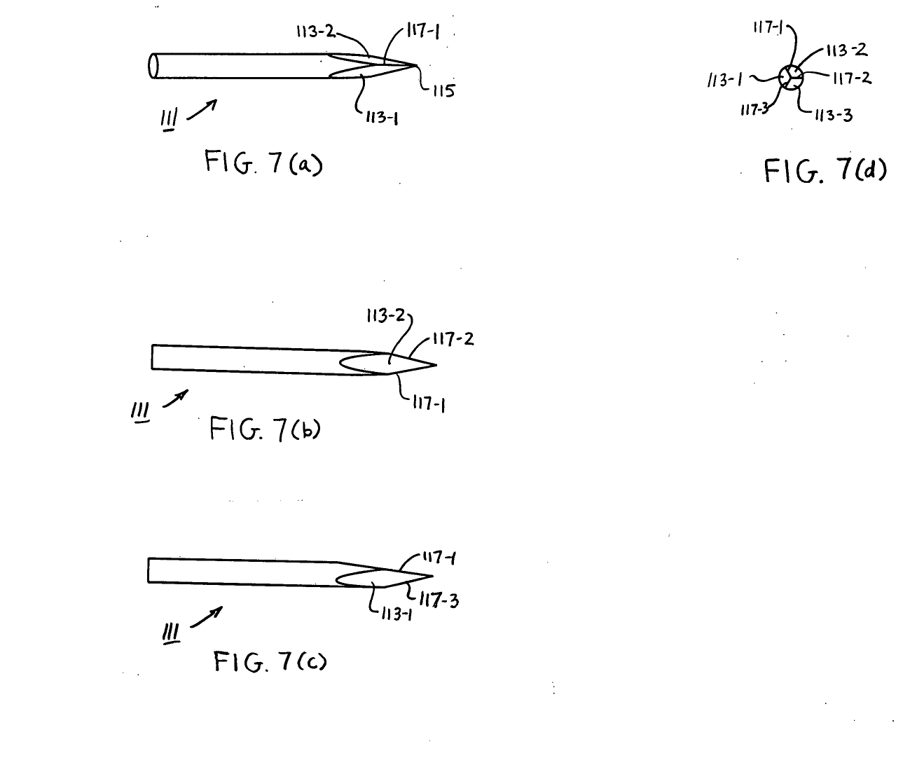

As an example, referring now to FIGS. 7(a)-(d), there is shown a sixth embodiment of a lancet constructed according to the teachings of the present invention, the lancet being identified generally by reference numeral 111. Lancet 111 is preferably formed by subjecting a solid (i.e., non-hollow) cylindrical tube which has a uniform circular cross-section along its length to three grinding processes.

Specifically, the three grinding processes are performed at approximately 120 degrees apart from one another about the longitudinal axis for lancet 111, each grind being performed at a planar angle of 7 degrees relative to the horizontal plane. The three grinding processes serve to create three adjacent single angle ground surfaces 113-1,113-2, 113-3 which together define a single sharpened tip 115.

It should be noted that adjacent ground surfaces 113 define an elongated cutting surface 117 therebetween. Accordingly, ground surfaces 113 serve to define first, second and third cutting surfaces 117-1, 117-2 and 117-3 which are spaced approximately 120 degrees apart from one another about the longitudinal axis for lancet 111. As can be appreciated, the formation of three separate cutting surfaces (as opposed to conventional lancets which only include two separate cutting surfaces) increases the likelihood that lancet 111 will puncture a capillary when fired into the skin of a patient, which is highly desirable.

It should also be noted that three additional grinding processes could be performed to lancet 111 to shorten the length of its sharpened tip, thereby rendering it more robust (i.e., strong). Specifically, fourth, fifth and sixth grinding processes can be performed with the unitary member disposed in the same orientation in which the first, second and third grinding processes are performed, wherein each of the three additional grinding processes is performed at an angle greater than the grinding angle of the three primary grinding processes.

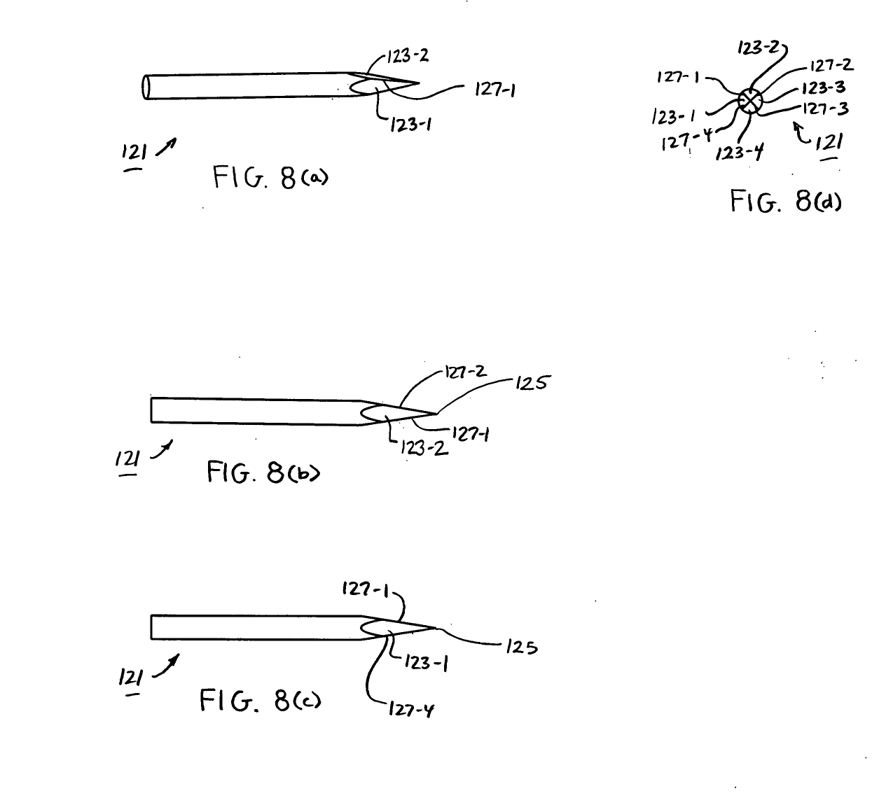

Referring now to FIGS. 8(a)-(d), there is shown a seventh embodiment of a lancet constructed according to the teachings of the present invention, the lancet being identified generally by reference numeral 121. Lancet 121 is preferably formed by subjecting a solid cylindrical tube which has a uniform circular cross-section along its length to four grinding processes.

Specifically, the four grinding processes are performed approximately 90 degrees apart from one another about the longitudinal axis for lancet 121, each grind being performed at a planar angle of 9 degrees relative to the horizontal plane. The four grinding processes serve to create four adjacent single angle ground surfaces 123-1, 123-2, 123-3 and 123-4 which together define a single sharpened tip 125.

It should be noted that adjacent ground surfaces 123 define an elongated cutting surface 127 therebetween. Accordingly, ground surfaces 123 serve to define first, second, third and fourth cutting surfaces 127-1, 127-2, 127-3 and 127-4 which are spaced approximately 90 degrees apart from one another about the longitudinal axis for lancet 121. As can be appreciated, the formation of four separate cutting surfaces (as opposed to conventional lancets which only include two separate cutting surfaces) increases the likelihood that lancet 121 will puncture a capillary when fired into the skin of a patient, which is highly desirable.

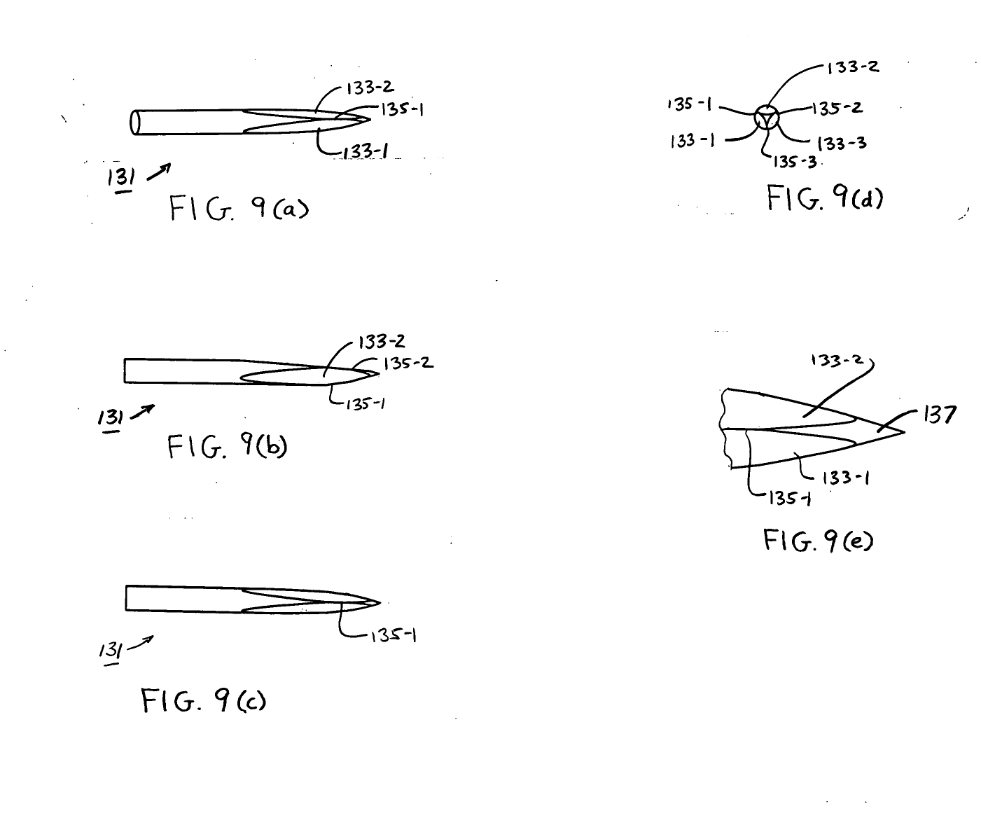

Referring now to FIGS. 9(a)-(e), there is shown an eighth embodiment of a lancet constructed according to the teachings of the present invention, the lancet being identified generally by reference numeral 131. Lancet 131 is preferably formed by subjecting a solid cylindrical tube which has a uniform circular cross-section along its length to four grinding processes.

Specifically, the first three grinding processes are performed at approximately 120 degrees apart from one another about the longitudinal axis for lancet 131, each grind being performed at a planar angle of 4 degrees relative to the horizontal plane. The first three grinding processes serve to create three adjacent single angle ground surfaces 133-1,133-2 and 133-3 which together define a single sharpened tip.

It should be noted that adjacent ground surfaces 133 define an elongated cutting surface 135 therebetween. Accordingly, ground surfaces 133 serve to define first, second and third cutting surfaces 135-1, 135-2 and 135-3 which are spaced approximately 120 degrees apart from one another about the longitudinal axis for lancet 131. As can be appreciated, the formation of three separate cutting surfaces (as opposed to conventional lancets which only include two separate cutting surfaces) increases the likelihood that lancet 131 will puncture a capillary when fired into the skin of a patient, which is highly desirable.

The fourth (and final) grinding process is performed on the sharpened tip. Specifically, a 32 degree cone-shaped grinding process is performed on the sharpened tip to create a shortened, rounded, ground-off tip 137, as seen most clearly in FIG. 9(e). As can be appreciated, the fourth grinding process serves to minimize the length of tip 137 to improve its strength. It should be noted that a cone-shaped grinding process is accomplished by drawing the lancet tip into contact against a grinding wheel with the longitudinal axis of the lancet disposed at a non-orthogonal angle relative to the grinding surface of said wheel and, at the same time, continuously rotating the lancet 360 degrees about its longitudinal axis.

The embodiments shown in the present invention are intended to be merely exemplary and those skilled in the art shall be able to make numerous variations and modifications to it without departing from the spirit of the present invention. All such variations and modifications are intended to be within the scope of the present invention as defined in the appended claims.

1. A lancet comprising:
(a) a seamless unitary member which is hollowed along at least a portion of its length, said unitary member including a first end, a second end, and a longitudinal axis,
(b) wherein the first end of said unitary member is shaped to include first and second sharpened tips.

2. The lancet as claimed in claim 1 wherein said seamless unitary member is in the form of a cylindrical tube.

3. A lancet comprising:
(a) a unitary member which is hollowed along at least a portion of its length, said unitary member including a first end and a second end,
(b) wherein the first end of said unitary member includes first and second ground surfaces which at least partially define first and second sharpened tips.

4. The lancet as claimed in claim 3 wherein said unitary member is in the form of a cylindrical tube.

5. The lancet as claimed in claim 3 wherein said unitary member includes a longitudinal axis, a lateral axis, a vertical axis, a horizontal plane and a vertical plane.

6. The lancet as claimed in claim 5 wherein each of said first and second ground surfaces is a planar surface.

7. The lancet as claimed in claim 6 wherein each of said first and second ground surfaces is a single angle ground surface.

8. The lancet as claimed in claim 7 wherein said first ground surface extends from the horizontal plane at an acute angle relative to the longitudinal axis.

9. The lancet as claimed in claim 8 wherein said first and second ground surfaces mirror one another about the horizontal plane.

10. The lancet as claimed in claim 9 wherein each of said first and second ground surfaces extends approximately 180 degrees about the longitudinal axis.

11. A method of manufacturing a lancet, said method comprising the steps of:
(a) providing a unitary member, said unitary member including a first end and a second end, and
(b) performing first and second grinds on the first end of said unitary member so as to yield first and second ground surfaces in the first end of said unitary member, said first and second ground surfaces at least partially defining first and second sharpened tips.

12. The method as claimed in claim 11 wherein said unitary member is in the form of a cylindrical tube.

13. The method as claimed in claim 12 wherein said unitary member is hollowed along at least a portion of its length.

14. The method as claimed in claim 11 further comprising the step of forming a longitudinal bore in said unitary member along at least a portion of its length.

15. The method as claimed in claim 11 wherein said unitary member includes a longitudinal axis, a lateral axis, a vertical axis, a horizontal plane and a vertical plane.

16. The method as claimed in claim 15 wherein each of said first and second ground surfaces is a planar surface.

17. The method as claimed in claim 16 wherein each of said first and second ground surfaces is a single angle ground surface.

18. The method as claimed in claim 17 wherein said first ground surface extends from the horizontal plane at a first acute angle relative to the longitudinal axis.

19. The method as claimed in claim 18 wherein said first and second ground surfaces mirror one another about the horizontal plane.

20. The method as claimed in claim 19 wherein each of said first and second ground surfaces extends approximately 180 degrees about longitudinal axis.

21. The method as claimed in claim 15 further comprising the steps of:
(a) performing third grind on the first end of said unitary member, said third grind creating a third ground surface in the first end of said unitary member, and
(b) performing a fourth grind on the first end of said unitary member, said fourth grind creating a fourth ground surface in the first end of said unitary member, said first and fourth ground surfaces at least partially defining the first sharpened tip and said second and third ground surfaces at least partially defining the second sharpened tip.

22. The method as claimed in claim 21 wherein each of said first, second, third and fourth ground surfaces is a planar surface.

23. The method as claimed in claim 22 wherein each of said first, second, third and fourth ground surfaces is a compound angle ground surface.

24. The method as claimed in claim 23 wherein said first ground surface extends from the horizontal plane at a first acute angle relative to the longitudinal axis and at a second acute angle relative to the lateral axis.

25. The method as claimed in claim 24 wherein said first and second ground surfaces mirror one another about the vertical plane, said second and third ground surfaces mirror one another about the horizontal plane, and said third and fourth ground surfaces mirror one another about the vertical plane.

26. The method as claimed in claim 25 wherein each of said first, second, third and fourth ground surfaces extends approximately 90 degrees about the longitudinal axis.

27. The method as claimed in claim 20 further comprising the steps of:
(a) performing a third grind on said first sharpened tip, said third grind creating a third ground surface in said first sharpened tip,
(b) performing a fourth grind on said first sharpened tip, said fourth grind creating a fourth ground surface in said first sharpened tip,
(c) performing a fifth grind on said second sharpened tip, said fifth grind creating a fifth ground surface in said second sharpened tip, and
(d) performing a sixth grind on said second sharpened tip, said sixth grind creating a sixth ground surface in said second sharpened tip.

28. The method as claimed in claim 27 wherein each of said third, fourth, fifth and sixth ground surfaces is a planar surface.

29. The method as claimed in claim 28 wherein each of said third, fourth, fifth and sixth ground surfaces is a compound angle ground surface.

30. The method as claimed in claim 29 wherein said third ground surface extends from the horizontal plane at a second acute angle relative to the longitudinal axis and at a third acute angle relative to the lateral axis.

31. The method as claimed in claim 30 wherein said third and fourth ground surfaces mirror one another about the horizontal plane, said third and fifth ground surfaces mirror one another about the vertical plane, and said fifth and sixth ground surfaces mirror one another about the horizontal plane.

32. A lancet comprising:
(a) a unitary member which is hollowed along at least a portion of its length, said unitary member including a first end and a second end,
(b) wherein the first end of said unitary member is shaped to include first, second and third sharpened tips.

33. The lancet of claim 32 wherein said unitary member is seamless.

34. The lancet of claim 33 wherein said unitary member is in the form of a cylindrical tube.

35. The lancet of claim 34 wherein the first end of said unitary member includes first, second and third ground surfaces.

36. The lancet as claimed in claim 35 wherein said unitary member includes a longitudinal axis, a lateral axis, a vertical axis, a horizontal plane and a vertical plane.

37. The lancet as claimed in claim 36 wherein each of said first, second and third ground surfaces is a planar surface.

38. The lancet as claimed in claim 37 wherein each of said first, second and third ground surfaces is a single angle ground surface.

39. The lancet as claimed in claim 38 wherein said first ground surface extends from the horizontal plane at an acute angle relative to the longitudinal axis.

40. The lancet as claimed in claim 39 wherein each of said first, second and third ground surfaces extends approximately 120 degrees about the longitudinal axis.

41. The lancet as claimed in claim 40 wherein said first, second and third ground surfaces are spaced equidistantly apart from one another.

42. A method of manufacturing a lancet, said method comprising the steps of:
(a) providing a unitary member, said unitary member including a first end and a second end, and
(b) performing first, second and third grinds on the first end of said unitary member so as to yield first, second and third ground surfaces in the first end of said unitary member, said first, second and third ground surfaces at least partially defining first, second and third sharpened tips in said unitary member.

43. The method as claimed in claim 42 wherein said unitary member is in the form of a cylindrical tube.

44. The method as claimed in claim 43 wherein said unitary member is hollowed along at least a portion of its length.

45. The method as claimed in claim 42 further comprising the step of forming a longitudinal bore in said unitary member along at least a portion of its length.

46. The method as claimed in claim 42 wherein said unitary member includes a longitudinal axis, a lateral axis, a vertical axis, a horizontal plane and a vertical plane.

47. The method as claimed in claim 46 wherein each of said first, second and third ground surfaces is a planar surface.

48. The method as claimed in claim 47 wherein each of said first, second and third ground surfaces is a single angle ground surface.

49. The method as claimed in claim 48 wherein said first ground surface extends from the horizontal plane at a first acute angle relative to the longitudinal axis.

50. The method as claimed in claim 49 wherein said first, second and third ground surfaces are spaced equidistantly apart from one another.

51. The method as claimed in claim 50 wherein each of said first, second and third ground surfaces extends approximately 120 degrees about the longitudinal axis.

52. The method as claimed in claim 46 further comprising the step of shortening said first, second and third sharpened tips.

53. The method as claimed in claim 52 wherein said step of shortening said first, second and third sharpened tips is accomplished by performing fourth, fifth and sixth grinds on the first end of said unitary member.

54. A lancet comprising:
(a) a unitary member including a first end, a second end and a longitudinal axis,
(b) wherein the first end of said unitary member is shaped to include first, second and third ground surfaces which together at least partially define a single tip and first, second and third cutting edges.

55. The lancet of claim 54 wherein the first, second and third cutting edges are spaced approximately 120 degrees apart from one another about the longitudinal axis.

56. A method of manufacturing a lancet, said method comprising the steps of:
(a) providing a unitary member which includes a first end, a second end and a longitudinal axis, and
(b) performing three separate grinds on the first end of said unitary member to yield first, second and third cutting edges which at least partially define a single sharpened tip.

57. The method of claim 56 wherein said first, second and third cutting edges are spaced approximately 120 degrees apart from one another about the longitudinal axis.

58. The method of claim 57 further comprising the step of performing a cone-shaped grind on the single sharpened tip to create a shortened, rounded, ground-off tip.

59. A lancet comprising:
(a) a unitary member including a first end, a second end and a longitudinal axis,
(b) wherein the first end of said unitary member is shaped to include first, second, third and fourth ground surfaces which together at least partially define a single tip and first, second, third and fourth cutting edges.

60. The lancet of claim 59 wherein the first, second, third and fourth cutting edges are spaced approximately 90 degrees apart from one another about the longitudinal axis.

61. A method of manufacturing a lancet, said method comprising the steps of:
(a) providing a unitary member which includes a first end, a second end and a longitudinal axis, and
(b) performing four separate grinds on the first end of said unitary member to yield first, second, third and fourth cutting edges which at least partially define a single sharpened tip.

63. The method of claim 62 wherein said first, second, third and fourth cutting edges are spaced approximately 90 degrees apart from one another about the longitudinal axis.
